(19) United States
(12) Patent Application Publication
(10) Pub. No.: US 2018/0042764 A1
(43) Pub. Date: Feb. 15, 2018

(54) METHODS FOR TREATMENT OF NON-GENITAL CUTANEOUS WARTS

(71) Applicant: Ferndale Laboratories, Inc., Ferndale, MI (US)

(72) Inventors: Israel Dvoretzky, Ferndale, MI (US); Michael Burns, Ferndale, MI (US)

(21) Appl. No.: 15/674,182

(22) Filed: Aug. 10, 2017

Related U.S. Application Data
(60) Provisional application No. 62/373,103, filed on Aug. 10, 2016.

(51) Int. Cl. A61F 7/03; A61K 31/4745; A61K 9/70; A61K 9/00; A61K 47/02; A61K 31/122; A61K 41/00

(52) U.S. Cl. CPC A61F 7/034; A61K 31/122; A61K 31/4745; A61K 41/0052; A61K 9/0014; A61K 47/02; A61K 9/7007; A61F 2007/036; A61F 2007/0263

(57) ABSTRACT

Methods of treating a wart are provided which include: providing a heating device having a central zone and peripheral zone, sized to cover the wart and skin peripheral to the wart, and including a heat-generating material that undergoes an exothermic reaction when exposed to oxygen; providing a liquid therapeutic adhesive comprising a volatile solvent, skin-compatible adhesive and therapeutic agent; applying the liquid therapeutic adhesive to the skin peripheral to the wart or to the peripheral zone of the heating device; initiating the exothermic reaction of the heat-generating material by exposure of the heat-generating material to oxygen; applying the central zone of the heating device to contact the wart, thereby heating the wart and skin peripheral to the wart to an elevated skin temperature; and maintaining the elevated skin temperature for a period of time, thereby creating an enclosed heated environment treating the wart and skin peripheral to the wart.

### REFERENCE TO RELATED APPLICATION

This application claims priority to U.S. Provisional Patent Application Ser. No. 62/373,103, filed Aug. 10, 2016, the entire content of which is incorporated herein by reference.

### FIELD OF THE INVENTION

The present invention relates generally to methods for treatment of warts. In specific aspects, the present invention relates to methods for treatment of non-genital cutaneous warts.

### BACKGROUND OF THE INVENTION

Non-genital cutaneous human papilloma virus infections are widespread. Prior methods of treatment have not eliminated the problem. There is a continuing need for methods of treatment of non-genital cutaneous warts.

### SUMMARY OF THE INVENTION

Methods of treating a wart and an area of skin peripheral to the wart in a subject in need thereof are provided according to aspects of the present invention which include: providing a heating device having a central zone and a peripheral zone, the heating device sized to cover at least the wart and the skin peripheral to the wart, and the heating device comprising a heat-generating material that undergoes an exothermic reaction when exposed to oxygen; providing a liquid therapeutic adhesive comprising a volatile solvent, a skin-compatible adhesive and a therapeutic agent; applying the liquid therapeutic adhesive composition to at least the skin peripheral to the wart or to at least the peripheral zone of the heating device; initiating the exothermic reaction of the heat-generating material by exposure of the heat-generating material to oxygen; applying the central zone of the heating device to contact the wart, whereby the peripheral zone of the heating device is positioned in indirect contact with the area of skin peripheral to the wart via the liquid therapeutic adhesive composition disposed on the skin peripheral to the wart or on the peripheral zone of the heating device, thereby adhering the heating device to at least the skin peripheral to the wart and thereby heating the wart and skin peripheral to the wart to an elevated skin temperature; and maintaining the elevated skin temperature for a period of time by leaving the heating device adhered to the skin peripheral to the wart until the exothermic reaction is completed, thereby creating an enclosed heated environment treating the wart and a heated therapeutic environment treating the skin peripheral to the wart.

Methods of treating a wart and an area of skin peripheral to the wart are provided according to aspects of the present invention which include applying the liquid therapeutic adhesive to the wart and/or central zone of the heating device.

Methods of treating a wart and an area of skin peripheral to the wart are provided according to aspects of the present invention, wherein the liquid therapeutic adhesive is not applied to the wart and/or central zone of the heating device.

Methods of treating a wart and an area of skin peripheral to the wart are provided according to aspects of the present invention, wherein the area of skin peripheral to the wart extends at least 0.1 cm radially outward from the peripheral border of the wart.

Methods of treating a wart and an area of skin peripheral to the wart are provided according to aspects of the present invention, wherein the area of skin peripheral to the wart extends from 0.1 cm-5 cm radially outward from the peripheral border of the wart.

Methods of treating a wart and an area of skin peripheral to the wart are provided according to aspects of the present invention, wherein the heat-generating material comprises iron.

Methods of treating a wart and an area of skin peripheral to the wart are provided according to aspects of the present invention, wherein the heat-generating material comprises iron powder.

Methods of treating a wart and an area of skin peripheral to the wart are provided according to aspects of the present invention, wherein the elevated skin temperature is in the range of 38° C.-45° C.

Methods of treating a wart and an area of skin peripheral to the wart are provided according to aspects of the present invention, wherein the elevated skin temperature is in the range of 42° C.-43° C.

Methods of treating a wart and an area of skin peripheral to the wart are provided according to aspects of the present invention, wherein the elevated skin temperature is maintained for at least a time in the range of 1 hour-24 hours.

Methods of treating a wart and an area of skin peripheral to the wart are provided according to aspects of the present invention, wherein the elevated skin temperature is maintained for at least a time in the range of 2 hours-12 hours.

Methods of treating a wart and an area of skin peripheral to the wart are provided according to aspects of the present invention, wherein the therapeutic agent is selected from the group consisting of: a keratolytic agent, a skin exfoliating material, a desquamating agent, an immunomodulator, a contact sensitizing agent, a wart treatment agent, a targeted immune therapy, a biologic drug, and a combination of any two or more thereof.

Methods of treating a wart and an area of skin peripheral to the wart are provided according to aspects of the present invention, wherein the therapeutic agent is selected from the group consisting of: a beta-hydroxy acid, an alpha-hydroxy acid, a retinoid, and a combination of any two or more thereof.

Methods of treating a wart and an area of skin peripheral to the wart are provided according to aspects of the present invention, wherein the therapeutic agent is selected from the group consisting of: salicylic acid, glycolic acid, lactic acid, retinol, urea, trichloroacetic acid, resorcinol, papain and a combination of any two or more thereof.

Methods of treating a wart and an area of skin peripheral to the wart are provided according to aspects of the present invention, wherein the therapeutic agent is a wart treatment agent selected from the group consisting of: podophyllotoxin, podophyllum resin, 5-fluorouracil, cantharone, a tar product, anthralin, benzoyl peroxide and a combination of any two or more thereof.

Methods of treating a wart and an area of skin peripheral to the wart are provided according to aspects of the present invention, wherein the therapeutic agent is squaric acid.

Methods of treating a wart and an area of skin peripheral to the wart are provided according to aspects of the present invention, wherein the therapeutic agent is imiquimod.

Methods of treating a wart and an area of skin peripheral to the wart are provided according to aspects of the present invention, wherein the therapeutic agent is selected from the group consisting of: salicylic acid, glycolic acid, lactic acid, retinol, urea, trichloroacetic acid, resorcinol, papain, podophyllotoxin, podophyllum resin, 5-fluorouracil, cantharone, a tar product, anthralin, benzoyl peroxide, squaric acid, imiquimod, and a combination of any two or more thereof.

Methods of treating a wart and an area of skin peripheral to the wart are provided according to aspects of the present invention, wherein the subject is a child, an adolescent, or an adult.

Methods of treating a wart and an area of skin peripheral to the wart in a subject in need thereof are provided according to aspects of the present invention which include: providing a heating device having a central zone and a peripheral zone, the heating device sized to cover at least the wart and the skin peripheral to the wart, the heating device comprising a heat-generating material that undergoes an exothermic reaction when exposed to oxygen, and the heating device comprising a layer of a non-therapeutic adhesive disposed on at least the peripheral zone of the heating device; providing a liquid therapeutic adhesive comprising a volatile solvent, a skin-compatible adhesive and a therapeutic agent; applying the liquid therapeutic adhesive composition to at least the skin peripheral to the wart or to at least the peripheral zone of the heating device; initiating the exothermic reaction of the heat-generating material by exposure of the heat-generating material to oxygen; applying the central zone of the heating device to contact the wart, whereby the peripheral zone of the heating device is positioned in indirect contact with the area of skin peripheral to the wart via the liquid therapeutic adhesive composition disposed on the skin peripheral to the wart or on the peripheral zone of the heating device, thereby adhering the heating device to at least the skin peripheral to the wart and thereby heating the wart and skin peripheral to the wart to an elevated skin temperature; and maintaining the elevated skin temperature for a period of time by leaving the heating device adhered to the skin peripheral to the wart until the exothermic reaction is completed, thereby creating an enclosed heated environment treating the wart and a heated therapeutic environment treating the skin peripheral to the wart.

Methods of treating a wart and an area of skin peripheral to the wart in a subject in need thereof are provided according to aspects of the present invention which include: providing a heating device having a central zone and a peripheral zone, the heating device sized to cover at least the wart and the skin peripheral to the wart, the heating device comprising a heat-generating material that undergoes an exothermic reaction when exposed to oxygen, and the heating device comprising a layer of a non-therapeutic adhesive disposed on the peripheral zone and the central zone of the heating device; providing a liquid therapeutic adhesive comprising a volatile solvent, a skin-compatible adhesive and a therapeutic agent; applying the liquid therapeutic adhesive composition to at least the skin peripheral to the wart or to at least the peripheral zone of the heating device; initiating the exothermic reaction of the heat-generating material by exposure of the heat-generating material to oxygen; applying the central zone of the heating device to contact the wart, whereby the peripheral zone of the heating device is positioned in indirect contact with the area of skin peripheral to the wart via the liquid therapeutic adhesive composition disposed on the skin peripheral to the wart or on the peripheral zone of the heating device, thereby adhering the heating device to at least the skin peripheral to the wart and thereby heating the wart and skin peripheral to the wart to an elevated skin temperature; and maintaining the elevated skin temperature for a period of time by leaving the heating device adhered to the skin peripheral to the wart until the exothermic reaction is completed, thereby creating an enclosed heated environment treating the wart and a heated therapeutic environment treating the skin peripheral to the wart.

### BRIEF DESCRIPTION OF THE DRAWINGS

FIG. 1 is a drawing illustrating a skin-facing side of a heating device used according to aspects of the present invention;

FIG. 2 is a drawing illustrating an opposing side of a heating device of FIG. 1 used according to aspects of the present invention;

FIG. 3 is a drawing illustrating a skin-facing side of a heating device used according to aspects of the present invention;

FIG. 4 is a drawing illustrating a cross-section of a heating device of FIG. 2 used according to aspects of the present invention;

FIG. 5 is a drawing illustrating a skin-facing side of a heating device used according to aspects of the present invention;

FIG. 6 is a drawing illustrating an opposing side of a heating device of FIG. 5 used according to aspects of the present invention;

FIG. 7 is a drawing illustrating a cross-section of a heating device of FIG. 6 used according to aspects of the present invention; and

FIG. 8 is a drawing illustrating aspects of a method of treating warts according to aspects of the present invention.

### DETAILED DESCRIPTION OF THE INVENTION

The singular terms “a,” “an,” and “the” are not intended to be limiting and include plural referents unless explicitly stated otherwise or the context clearly indicates otherwise.

Methods for treating a wart and an area of skin peripheral to the wart are provided according to aspects of the present invention which include providing a heating device having a central zone and a peripheral zone and sized to cover at least the wart and the area of skin peripheral to the wart. The heating device includes a heat-generating material that undergoes an exothermic reaction when exposed to oxygen.

According to preferred aspects, the heating device is larger than the wart to be treated and the heating device treats skin peripheral to the wart to inhibit spread and recurrence of the wart.

According to aspects of the present invention, a liquid therapeutic adhesive including a volatile solvent, a skin-compatible adhesive and a therapeutic agent is applied by a user having a wart to at least the area of skin peripheral to the wart or to at least the peripheral zone of the heating device.

According to aspects of the present invention, a liquid therapeutic adhesive including a volatile solvent, a skin-compatible adhesive and a keratolytic agent is applied by a user having a wart to at least the area of skin peripheral to the wart or to at least the peripheral zone of the heating device.

According to aspects of the present invention, a liquid therapeutic adhesive including a volatile solvent, a skin-compatible adhesive and salicylic acid is applied by a user having a wart to at least the area of skin peripheral to the wart or to at least the peripheral zone of the heating device.

According to particular aspects of the present invention, the liquid therapeutic adhesive is applied only to the area of skin peripheral to the wart or the peripheral zone of the heating device and is not applied to the wart or central zone of the heating device. Application of the liquid therapeutic adhesive only to the area of skin peripheral to the wart or the peripheral zone of the heating device is found to create a highly desirable heated therapeutic environment in the skin peripheral to the wart and superior therapeutic effects may be afforded when the liquid therapeutic adhesive is not applied to the wart directly or by contact with the liquid therapeutic adhesive on the central zone of the heating device. Further, a chamber effect at the site of the wart is provided by application of the liquid therapeutic adhesive only to the area of skin peripheral to the wart or the peripheral zone of the device without application of the liquid therapeutic adhesive directly to the wart, wherein an improved heat profile is present in the chamber. Still further, application of heat at the site of application of the liquid therapeutic adhesive to the skin peripheral to the wart improves penetration of the keratolytic agent.

The exothermic reaction of the heat-generating material is initiated by exposure of the heat-generating material to oxygen and the central zone of the heating device is applied to contact the wart, thus also positioning the peripheral zone of the heating device. The peripheral zone of the heating device is in contact with the area of skin peripheral to the wart indirectly since the liquid therapeutic adhesive composition is disposed on the skin peripheral to the wart or on the peripheral zone of the heating device. The heating device is thus adhered to at least the skin peripheral to the wart by the liquid therapeutic adhesive, or adhered exclusively to the skin peripheral to the wart by the liquid therapeutic, and the wart and skin peripheral to the wart are heated to an elevated skin temperature.

The elevated skin temperature is maintained for a period of time by leaving the heating device adhered to the skin peripheral to the wart until the exothermic reaction is completed, thereby creating an enclosed heated environment treating the wart and a heated therapeutic environment treating the skin peripheral to the wart.

The term “wart” as used herein refers to non-genital cutaneous benign skin growths caused by a human papilloma virus, including “common” warts (verruca vulgaris) and plantar warts as well as less common non-genital cutaneous warts such as filiform warts, plane warts (verruca plana), deep palmoplantar warts (myrmecia) and Butcher's warts.

The term “treating” is used herein to refer to eliminating a wart by the inventive method as well as inhibiting growth and/or spreading of the wart.

The phrase “skin peripheral to the wart” refers to skin of an area to be treated defined as extending radially outward from the peripheral border of the wart, wherein the area has an outer perimeter typically in the range of 0.1 cm-5 cm from the peripheral border of the wart, although this can be smaller or larger if desired.

A heating device including a heat-generating material that undergoes an exothermic reaction when exposed to oxygen used in methods according to aspects of the present invention has a central zone and a peripheral zone wherein the central zone is applied to cover and heat the wart and the peripheral zone is applied simultaneously to cover and heat an area of peripheral skin. The central and peripheral zones of the heating device are generally defined by the size of the user's wart, that is, the central zone of the heating device is approximately the same size as the wart and the peripheral zone of the heating device is approximately the same size as the skin peripheral to the wart.

Heating devices used in methods according to aspects of the present invention are configured to control initiation of the exothermic reaction by the heat-generating material. According to aspects of the invention, the initiation of the exothermic reaction by the heat-generating material is controlled by blocking exposure of the heat-generating material to oxygen until it is desired to initiate the exothermic reaction.

A liquid therapeutic adhesive used according to aspects of the present invention allows a user to create an enclosed heated environment bounded by the therapeutic adhesive at the location of the wart thereby treating the wart. Further, application of the liquid therapeutic adhesive to the skin peripheral to the wart creates a heated therapeutic environment in the skin peripheral to the wart and thereby provides a therapeutic effect in addition to the heat for inhibiting wart virus, including inhibition of infection of additional cells and/or inhibition of replication of the wart virus, in skin cells peripheral to the wart and thereby inhibiting the growth and spreading of the wart and wart virus.

According to aspects of the present invention, the liquid therapeutic adhesive includes a volatile solvent, a skin-compatible adhesive and a therapeutic agent.

The term “therapeutic agent” as used herein refers to a skin treatment agent that inhibits papilloma virus such as inhibition of infection of cells by human papilloma virus and/or inhibition of replication of human papilloma virus. Therapeutic agents include, but are not limited to, keratolytic agents, skin exfoliating materials, desquamating agents, immunomodulators, contact sensitizing agents, wart treatment agents and combinations of any two or more thereof. Examples of keratolytic agents, skin exfoliating materials and desquamating agents include, but are not limited to, salicylic acid and other beta-hydroxy acids, glycolic acid, lactic acid and other alpha-hydroxy acids, retinol and other retinoids, urea and related compounds, trichloroacetic acid, resorcinol and related compounds, papain and other enzymes. Wart treatment agents include, but are not limited to podophyllotoxin, podophyllum resin, 5-fluorouracil, cantharone, tar products, anthralin, benzoyl peroxide and combinations of any two or more thereof. Contact sensitizing agents are exemplified by, but not limited to, squaric acid. Immunomodulators are exemplified by, but not limited to, imiquimod and related compounds.

According to aspects of the present invention, the liquid therapeutic adhesive includes a volatile solvent, a skin-compatible adhesive and a keratolytic agent.

According to particular aspects of the present invention, the liquid therapeutic adhesive includes a volatile solvent, a skin-compatible adhesive and salicylic acid.

The volatile solvent can be any organic solvent which vaporizes at skin temperature of a living human, which does not degrade or otherwise interfere with the function of the skin-compatible adhesive to adhere the device to the skin and which does not degrade or otherwise interfere with the function of the therapeutic agent (e.g., salicylic acid) to inhibit the growth and spreading of the wart and wart virus. The volatile solvent is believed to enhance penetration of the therapeutic agent (e.g., salicylic acid) into cells infected with wart virus to provide an enhanced therapeutic effect. Examples of volatile solvents included in a liquid therapeutic adhesive according to aspects of the present invention include ethanol, 1-butanol, 2-butanol, isopropanol, 1-propanol, 2-propanol, benzyl alcohol, acetic acid, acetone, methyl acetate, ethyl acetate, butyl acetate, isobutyl acetate, propyl acetate, isopropyl acetate, tert-butylmethyl ether, ethyl ether, n-butyl ether, ethyl formate, formic acid, methylethyl ketone, methylisobutyl ketone, 2-methyl-1-propanol, isoparaffins, pentane, heptane, isopentane, isododecane, kerosene, xylene, silicones such as cyclomethicones and dimethicones, or a combination of any two or more thereof. An aqueous or non-volatile solvent can also be included in addition to the volatile solvent to optimize solubilization or provide other desirable properties if desired.

The term “skin-compatible adhesive” refers to a material that, when applied to the skin, does not cause substantial skin irritation over the period of treatment, typically 1-24 hours or less. Skin-compatible adhesives illustratively including acrylic polymers, silicones, hydrocolloids and/or polyurethanes can be used. According to particular aspects of the present invention, the skin-compatible adhesive includes gum mastic.

According to particular aspects of the present invention, a therapeutic agent is included in the liquid therapeutic adhesive in an amount in the range of 1-60 weight percent. Advantageously, the concentration of the therapeutic agent at the treatment site, skin peripheral to the wart and/or wart, is increased during treatment in view of the removal of the volatile solvent over time, enhancing the therapeutic effect.

According to particular aspects of the present invention, salicylic acid is included in the liquid therapeutic adhesive in an amount in the range of 1-60 weight percent. Advantageously, the concentration of salicylic acid, for example, at the treatment site, skin peripheral to the wart and/or wart, is increased during treatment in view of the removal of the volatile solvent over time enhancing the therapeutic effect.

One or more excipients are optionally included in a liquid therapeutic adhesive, such as, but not limited to, a preservative, a thickener, a buffer, a diluent, a colorant and a fragrance.

According to particular aspects of the present invention, the liquid therapeutic adhesive includes ethanol, gum mastic and a therapeutic agent. The ratio of ethanol:gum mastic:therapeutic agent is about 1-20 weight percent ethanol: 70-80 weight percent gum mastic: 1-60 weight percent therapeutic agent.

According to particular aspects of the present invention, the liquid therapeutic adhesive includes ethanol, gum mastic and salicylic acid. The ratio of ethanol:gum mastic:salicylic acid is about 1-20 weight percent ethanol: 70-80 weight percent gum mastic: 1-60 weight percent salicylic acid. According to particular aspects of the present invention, the ratio of ethanol:gum mastic:salicylic acid is about 10-20 weight percent ethanol: 70-80 weight percent gum mastic: 1-10 weight percent salicylic acid.

A heat-generating material that undergoes an exothermic reaction when exposed to oxygen according to aspects of the present invention includes a metal oxidizable by an oxidation reaction to generate heat, such as iron, aluminum, zinc, copper or mixtures thereof, preferably in the form of a metal powder, and one or more additional reactants or excipients for optimizing and controlling heat generation and distribution. According to particular aspects of the present invention, a water soluble salt is included in the heat-generating material as a catalyst of the oxidation reaction, such as an alkali metal chloride, alkaline earth metal chloride, alkali metal sulfate, alkaline earth metal sulfate, metal chloride or metal sulfate, non-limiting examples of which include sodium chloride, potassium chloride, calcium chloride, magnesium chloride, sodium sulfate, potassium sulfate, calcium sulfate and magnesium sulfate.

Water is included in the heat-generating material.

Optionally and preferably, a carbon material is included in the heat-generating material as a reaction accelerator, non-limiting examples of which include activated carbon, graphite, carbon black, charcoal and coal.

Further optionally and preferably, a water absorbent material is included in the heat-generating material to provide a reservoir for water and ensure its presence in the exothermic reaction mix, non-limiting examples of which include silica gel, vermiculite, wood pulp, perlite, aluminum oxide, and water-absorbent polymers.

General proportions of each component included in a heat-generating material are, for example, in the ranges of 30-80 weight percent oxidizable metal, preferably in the form of an oxidizable metal powder, 1-25 weight percent carbon material, 1-20 weight percent water absorbent material, 1-10 weight percent water soluble salt and 1-40 weight percent water. The heat-generating material is prepared by mixing the desired components in proportions to achieve the desired speed and extent of heat release and reaction duration. Mixing is preferably performed without significant exposure of the oxidizable metal to oxygen in the presence of other reactants, such as under nitrogen or other inert gas.

According to preferred aspects, the heat-generating material includes iron powder, a salt catalyst, a carbon material, a water absorbent material and water.

The heat-generating material is enclosed in a container, wherein at least a portion of the container is air permeable.

According to preferred aspects of the invention, the container is a flexible pouch wherein at least a portion of the flexible pouch is air permeable and adaptable to generally conform to the shape of the area of skin to be treated.

The air permeable portion of the container can be any air permeable material which retains the heat-generating material, such as an air permeable woven or non-woven natural, synthetic or semi-synthetic fabric. Non-limiting examples of substances included in air permeable woven or non-woven natural, synthetic or semi-synthetic fabrics are cotton, silk, cellulose, hemp, rayon, acrylic, nylon, polyethylene, polypropylene, acetate, polyester; and combinations of any two or more thereof.

The air permeable portion of the container is reversibly covered with an oxygen impermeable material so as to prevent contact of the heat-generating material with oxygen and thereby control the initiation of the oxidation reaction for heat-generation.

For example, the heating device is enclosed in an oxygen impermeable package prior to use to prevent contact with oxygen prior to use. The oxygen impermeable package can be filled with an inert gas, such as nitrogen.

In a further example, the air permeable portion of the container is reversibly covered with a removable layer of an oxygen impermeable material such as a sheet or film layer of polymer such as, but not limited to, acrylic, nylon, polyethylene, polypropylene, acetate, polyester, polyimide, polyurethane, polystyrene, polycarbonate, polyvinylidene chloride, ethylene-vinyl acetate copolymer, and mixtures of any two or more thereof. The sheet or film layer is reversibly adhered to the air permeable portion of the container and removed, for example, by peeling or tearing away to expose the air permeable portion.

According to aspects of the present invention, a portion of the heating device to be applied to the wart and skin peripheral to the wart is inert with respect to the liquid therapeutic adhesive, impermeable to the liquid therapeutic adhesive and capable of allowing conductance of heat from the heat-generating material to the wart and skin. The phrase “inert with respect to the liquid therapeutic adhesive” indicates that the integrity of the portion of the heating device to be applied to the wart is not affected by the liquid therapeutic adhesive at least during the period of treatment, typically 1-24 hours. This portion of the heating device can be, for example, a sheet layer or film of polymer such as, but not limited to, acrylic, nylon, polyethylene, polypropylene, acetate, polyester, polyimide, polyurethane, polystyrene, polycarbonate, polyvinylidene chloride, ethylene-vinyl acetate copolymer, and mixtures of any two or more thereof.

A non-therapeutic adhesive is optionally disposed on the portion of the heating device to be applied to the wart and skin peripheral to the wart to aid in adhering the heating device to the skin. The term “non-therapeutic adhesive” refers to a skin-compatible material capable of adhering to the skin of a user but which does not itself provide therapeutic benefit apart from functioning to aid in adhering the heating device to the user's skin. Any skin-compatible adhesive can be used.

A sheet layer or film of natural, synthetic or semi-synthetic polymer, paper is reversibly adhered to the non-therapeutic adhesive and is removed prior to use. Examples of polymers included in the sheet layer or film include, but are not limited to, paper, cellulose, acrylic, nylon, polyethylene, polypropylene, acetate, polyester, polyimide, polyurethane, polystyrene, polycarbonate, polyvinylidene chloride, ethylene-vinyl acetate copolymer, mixtures of any two or more thereof or layered combinations of any two or more thereof.

As indicated above, the user applies the liquid therapeutic adhesive composition to at least the area of skin peripheral to the wart or to at least the peripheral zone of the heating device. Upon initiating the exothermic reaction of the heat-generating material by exposure of the heat-generating material to oxygen, the central zone of the heating device is applied to contact the wart, and this simultaneously brings the peripheral zone of the heating device into position with the area of skin peripheral to the wart. The peripheral zone of the heating device is in indirect contact with the area of skin peripheral to the wart via the liquid therapeutic adhesive composition which was disposed on the skin peripheral to the wart or on the peripheral zone of the heating device, such that a layer of the liquid therapeutic adhesive is between the heating device and the skin. The heating device is thereby adhered to at least the skin peripheral to the wart. Optionally, the liquid therapeutic adhesive is also applied to the central zone of the heating device or to the wart. In a further option, the liquid therapeutic adhesive can be applied both to the heating device and the skin or wart.

Application of the heating device heats the wart and skin peripheral to the wart to an elevated skin temperature in the range of 38° C.-45° C., preferably 42° C.-43° C. The elevated skin temperature is maintained for a period of treatment time by leaving the heating device adhered to the skin peripheral to the wart until the exothermic reaction is completed. The treatment time is in the range of 1 hour-24 hours, preferably 2-12 hours. The treatment can be repeated one or more times, such as hourly, twice daily, daily, every other day, or weekly, until the desired effect is achieved.

The heating device is characterized by a “heating profile” in operation. The heating profile can be characterized by quick heating, e.g., 10-30 minutes, to a peak temperature which is maintained for a treatment time in the range of 1 hour-24 hours, preferably 2-12 hours, for example. Alternatively, the heating profile can be characterized by slow heating, e.g., over 2-3 hours, to a peak temperature which is maintained for a relatively short time, e.g., 15-30 minutes, followed by another period of, e.g., 2-3 hours during which the peak temperature decreases back to skin temperature.

Application of the heating device creates an enclosed heated environment treating the wart and a heated therapeutic environment treating the skin peripheral to the wart. The enclosed heated therapeutic environment promotes inhibition of the wart virus at the site of the wart. The heated therapeutic environment promotes inhibition of the wart virus in skin peripheral to the wart and promotes the effects of the therapeutic agent (e.g., salicylic acid) in the skin peripheral to the wart.

In a still further option, the heating device has a skin-compatible adhesive present on the portion to be applied to the skin and/or wart in addition to the liquid therapeutic adhesive.

A subject to be treated by a method of treating a wart and an area of skin peripheral to the wart according to aspects of the present invention can be any age or sex. The term “subject” as used herein refers to a human child, a human adolescent, or a human adult.

According to aspects of the present invention, the subject is a child and the heating device has a layer of material which is impermeable to air and liquids, including a central zone to be applied to a wart and a peripheral zone to be applied to skin peripheral to the wart, and having a removable sheet layer reversibly adhered to the layer of material which is impermeable to air and liquids by a non-therapeutic adhesive which aids in adhering the heating device to the skin. A liquid therapeutic adhesive including a volatile solvent, a skin-compatible adhesive and a therapeutic agent is applied to at least the skin peripheral to the wart of the child or to at least the peripheral zone of the heating device. The exothermic reaction of the heat-generating material is initiated by exposure of the heat-generating material to oxygen. The central zone of the heating device is applied to contact the wart of the child, whereby the peripheral zone of the heating device is positioned in indirect contact with the area of skin peripheral to the wart via the liquid therapeutic adhesive composition disposed on the skin peripheral to the wart or on the peripheral zone of the heating device, thereby adhering the heating device to at least the skin peripheral to the wart and thereby heating the wart and skin peripheral to the wart to an elevated skin temperature; and maintaining the elevated skin temperature for a period of time by leaving the heating device adhered to the skin peripheral to the wart until the exothermic reaction is completed, thereby creating an enclosed heated environment treating the wart and a heated therapeutic environment treating the skin peripheral to the wart. Optionally, the non-therapeutic adhesive is omitted.

According to aspects of the present invention, the subject is an adolescent and the heating device has a layer of material which is impermeable to air and liquids, including a central zone to be applied to a wart and a peripheral zone to be applied to skin peripheral to the wart, and having a removable sheet layer reversibly adhered to the layer of material which is impermeable to air and liquids by a non-therapeutic adhesive which aids in adhering the heating device to the skin. A liquid therapeutic adhesive including a volatile solvent, a skin-compatible adhesive and a therapeutic agent is applied to at least the skin peripheral to the wart of the adolescent or to at least the peripheral zone of the heating device. The exothermic reaction of the heat-generating material is initiated by exposure of the heat-generating material to oxygen. The central zone of the heating device is applied to contact the wart of the adolescent, whereby the peripheral zone of the heating device is positioned in indirect contact with the area of skin peripheral to the wart via the liquid therapeutic adhesive composition disposed on the skin peripheral to the wart or on the peripheral zone of the heating device, thereby adhering the heating device to at least the skin peripheral to the wart and thereby heating the wart and skin peripheral to the wart to an elevated skin temperature; and maintaining the elevated skin temperature for a period of time by leaving the heating device adhered to the skin peripheral to the wart until the exothermic reaction is completed, thereby creating an enclosed heated environment treating the wart and a heated therapeutic environment treating the skin peripheral to the wart. Optionally, the non-therapeutic adhesive is omitted.

According to aspects of the present invention, the subject is an adult and the heating device has a layer of material which is impeimeable to air and liquids, including a central zone to be applied to a wart and a peripheral zone to be applied to skin peripheral to the wart, and having a removable sheet layer reversibly adhered to the layer of material which is impermeable to air and liquids by a non-therapeutic adhesive which aids in adhering the heating device to the skin. A liquid therapeutic adhesive including a volatile solvent, a skin-compatible adhesive and a therapeutic agent is applied to at least the skin peripheral to the wart of the adult or to at least the peripheral zone of the heating device. The exothermic reaction of the heat-generating material is initiated by exposure of the heat-generating material to oxygen. The central zone of the heating device is applied to contact the wart of the adult, whereby the peripheral zone of the heating device is positioned in indirect contact with the area of skin peripheral to the wart via the liquid therapeutic adhesive composition disposed on the skin peripheral to the wart or on the peripheral zone of the heating device, thereby adhering the heating device to at least the skin peripheral to the wart and thereby heating the wart and skin peripheral to the wart to an elevated skin temperature; and maintaining the elevated skin temperature for a period of time by leaving the heating device adhered to the skin peripheral to the wart until the exothermic reaction is completed, thereby creating an enclosed heated environment treating the wart and a heated therapeutic environment treating the skin peripheral to the wart. Optionally, the non-therapeutic adhesive is omitted.

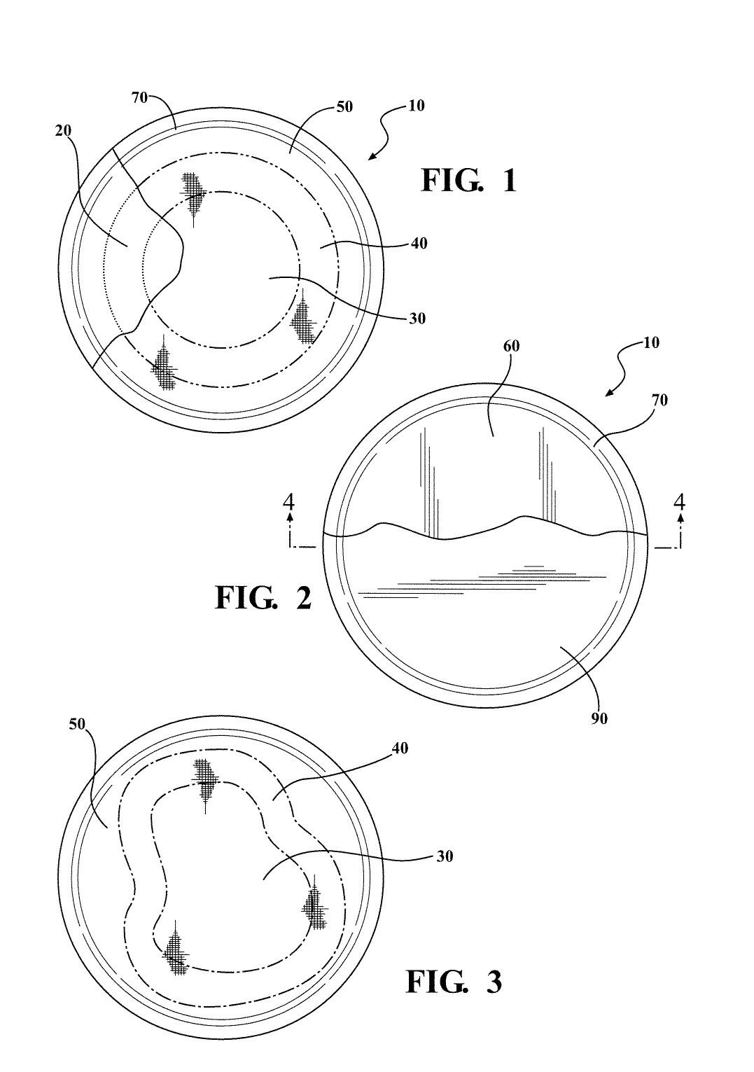

FIGS. 1-7 show aspects of a heating device used in methods according to aspects of the present invention wherein the heating device is configured as a pouch containing a heat-generating material that undergoes an exothermic reaction when exposed to oxygen, wherein the pouch is generally flat and has a first side formed of a layer of oxygen permeable material and an opposing second side formed of a layer of material which is impermeable to oxygen and liquids, the first and second sides affixed to each other to enclose the heat-generating material that undergoes an exothermic reaction when exposed to oxygen. In use, the second side is applied to the wart and skin peripheral to the wart.

FIG. 1 shows a view of a skin-facing side of a heating device 10, having a layer of material 50 which is impermeable to air and liquids, including a central zone 30 to be applied to a wart and a peripheral zone 40 to be applied to skin peripheral to the wart. A removable sheet layer 20 is shown reversibly adhered to layer 50 by a non-therapeutic adhesive which aids in adhering the heating device to the skin. The inclusion of the non-therapeutic adhesive is an option. The removable sheet layer 20 adhered to layer 50 is also an option and may be omitted if the non-therapeutic adhesive layer is omitted.

FIG. 2 shows a view of the opposing side of heating device 10, including a layer of air permeable material 60. As described herein, the air permeable material is reversibly covered with an oxygen impermeable material such an enclosed in an oxygen impermeable package or reversibly covered with a removable layer of an oxygen impermeable material until use so as to prevent contact of the heat-generating material with oxygen and thereby control the initiation of the oxidation reaction for heat-generation. FIG. 2 shows the air permeable material 60 reversibly covered with a removable layer of an oxygen impermeable material 90.

The layers 50 and 60 are joined at the periphery 70 of the device 10 to enclose the heat-generating material that undergoes an exothermic reaction when exposed to oxygen. Layers 50 and 60 can be joined by any of various joining methods such as adhesion and/or crimping.

Optionally, the central and peripheral zones on the side of the skin-facing side of a heat-generating device can be indicated by indicia included on the layer of material which is impermeable to oxygen and liquids 50. Alternatively, a user can be instructed about the general location of the central and peripheral zones and the user can adjust the location and size of the central and peripheral zones according to the particular wart treatment required.

FIG. 3 shows an irregularly shaped central zone 30 and peripheral zone 40 illustrating adaptation of the device to a particular size and shape of the user's wart. The liquid therapeutic adhesive is applied in the peripheral zone 40 directly on the device or can be applied to the peripheral zone 40 indirectly by applying the liquid therapeutic adhesive to the area of skin peripheral to the wart prior to application of the heating device.

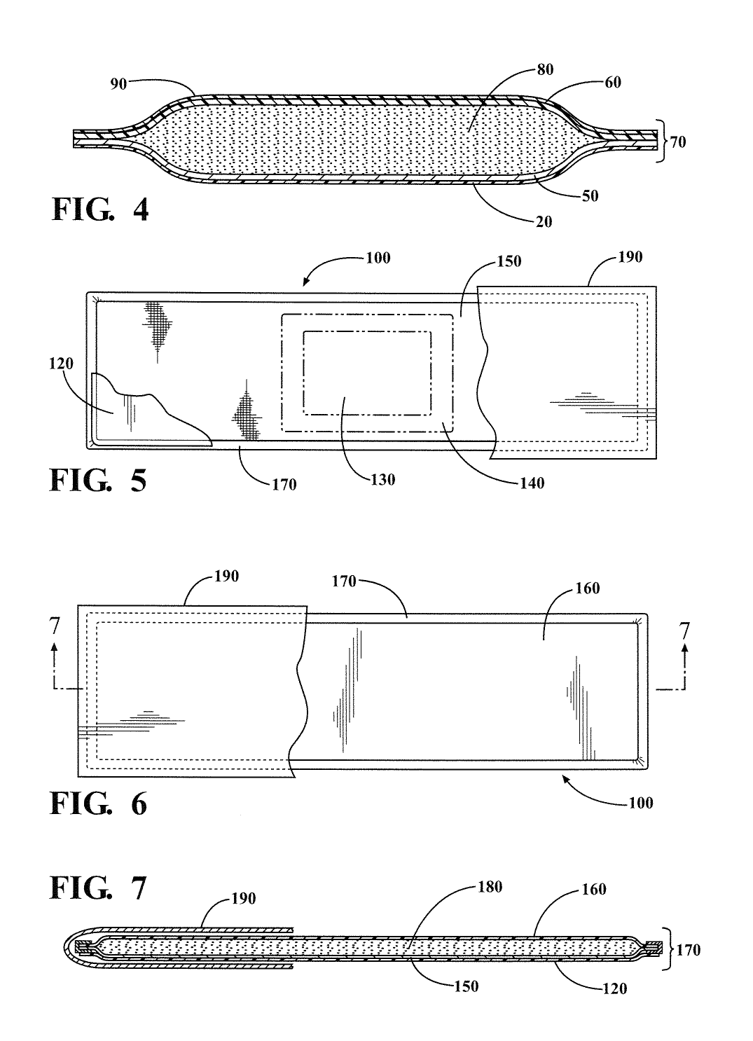

FIG. 4 is a cross-section of the device 10 shown at 4-4 in FIG. 2, illustrating removable adhered layer 20, a layer of oxygen permeable material 60 and a layer of material 50 which is impermeable to oxygen and liquids on the side to be applied to the wart and skin. A region 70 at the periphery joining the layers 50 and 60 is shown. Heat-generating material 80 is enclosed in the pouch formed by joinder of layers 50 and 60. A non-therapeutic adhesive is disposed between layers 20 and 50 providing adhesion to the skin in addition to the liquid therapeutic adhesive to be applied to the device and/or skin according to the illustrated embodiment. FIG. 4 shows the air permeable material 60 reversibly covered with a removable layer of an oxygen impermeable material 90.

FIG. 5 shows a view of a skin-facing side of a heating device 100, having removable adhered layer 120, a layer of material 150 which is impermeable to air and liquids, including a central zone 130 to be applied to a wart and a peripheral zone 140 to be applied to skin peripheral to the wart. FIG. 5 shows the device reversibly covered with a removable package of an oxygen impermeable material 190.

FIG. 6 shows a view of the opposing side of heating device 100, including a layer of air permeable material 160. A region 170 at the periphery of device 100 joining the layers 150 and 160 is shown. FIG. 6 shows the device reversibly covered with a removable package of an oxygen impermeable material 190.

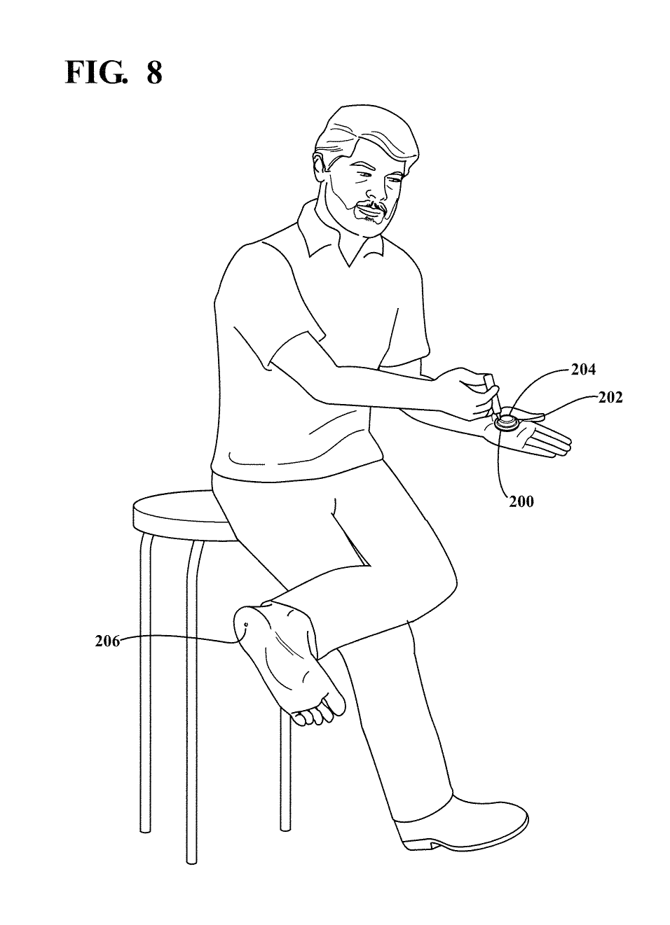

FIG. 7 is a cross-section of the device 100 shown at 7-7 in FIG. 5, illustrating removable adhered layer 120, a layer of oxygen permeable material 160, a layer of material 150 which is impermeable to oxygen and liquids on the side to be applied to the wart and skin and region 170 at the periphery joining the layers 150 and 160. FIG. 7 shows the device reversibly covered with a removable package of an oxygen impermeable material 190.

Heat-generating material 180 is enclosed in the pouch formed by joinder of layers 150 and 160. An adhesive is disposed between layers 120 and 150 which provides adhesion to the skin in addition to the liquid therapeutic adhesive according to the illustrated embodiment.

FIG. 8 illustrates an aspect of a method of treating a wart and an area of skin peripheral to the wart according to the present invention. A user is shown applying liquid therapeutic adhesive 202 to the peripheral zone of the heating device 200 prior to applying the central zone 204 of the device to the wart 206. Alternatively, the user applies the liquid therapeutic adhesive to an area of skin peripheral to the wart prior to applying the central zone 204 of the device to the wart 206.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for treating a wart and an area of skin peripheral to the wart of a subject, comprising:
providing a heating device having a central zone and a peripheral zone, the heating device sized to cover at least the wart and the skin peripheral to the wart, and the heating device comprising a heat-generating material that undergoes an exothermic reaction when exposed to oxygen;
providing a liquid therapeutic adhesive comprising a volatile solvent, a skin-compatible adhesive and a therapeutic agent;
applying the liquid therapeutic adhesive composition to at least the skin peripheral to the wart or to at least the peripheral zone of the heating device;
initiating the exothermic reaction of the heat-generating material by exposure of the heat-generating material to oxygen;
applying the central zone of the heating device to contact the wart, whereby the peripheral zone of the heating device is positioned in indirect contact with the area of skin peripheral to the wart via the liquid therapeutic adhesive composition disposed on the skin peripheral to the wart or on the peripheral zone of the heating device, thereby adhering the heating device to at least the skin peripheral to the wart and thereby heating the wart and skin peripheral to the wart to an elevated skin temperature; and
maintaining the elevated skin temperature for a period of time by leaving the heating device adhered to the skin peripheral to the wart until the exothermic reaction is completed, thereby creating an enclosed heated environment treating the wart and a heated therapeutic environment treating the skin peripheral to the wart.

2. The method of claim 1, further comprising applying the liquid therapeutic adhesive to the wart and/or central zone of the heating device.

3. The method of claim 1, wherein the liquid therapeutic adhesive is not applied to the wart and/or central zone of the heating device.

4. The method of claim 1, wherein the area of skin peripheral to the wart extends at least 0.1 cm radially outward from the peripheral border of the wart.

5. The method of claim 1, wherein the area of skin peripheral to the wart extends from 0.1 cm-5 cm radially outward from the peripheral border of the wart.

6. The method of claim 1, wherein the heat-generating material comprises iron.

7. The method of claim 1, wherein the heat-generating material comprises iron powder.

8. The method of claim 1, wherein the elevated skin temperature is in the range of 38° C.-45° C.

9. The method of claim 1, wherein the elevated skin temperature is in the range of 42° C.-43° C.

10. The method of claim 8, wherein the elevated skin temperature is maintained for at least a time in the range of 1 hour-24 hours.

11. The method of claim 10, wherein the elevated skin temperature is maintained for at least a time in the range of 2 hours-12 hours.

12. The method of claim 1, wherein the therapeutic agent is selected from the group consisting of: a keratolytic agent, a skin exfoliating material, a desquamating agent, an immunomodulator, a contact sensitizing agent, a wart treatment agent, a targeted immune therapy, a biologic drug, and a combination of any two or more thereof.

13. The method of claim 1, wherein the therapeutic agent is selected from the group consisting of: a beta-hydroxy acid, an alpha-hydroxy acid, a retinoid, and a combination of any two or more thereof.

14. The method of claim 1, wherein the therapeutic agent is selected from the group consisting of: salicylic acid, glycolic acid, lactic acid, retinol, urea, trichloroacetic acid, resorcinol, papain and a combination of any two or more thereof.

15. The method of claim 1, wherein the therapeutic agent is a wart treatment agent selected from the group consisting of: podophyllotoxin, podophyllum resin, 5-fluorouracil, cantharone, a tar product, anthralin, benzoyl peroxide and a combination of any two or more thereof.

16. The method of claim 1, wherein the therapeutic agent is squaric acid.

17. The method of claim 1, wherein the therapeutic agent is imiquimod.

18. The method of claim 1, wherein the therapeutic agent is selected from the group consisting of: salicylic acid, glycolic acid, lactic acid, retinol, urea, trichloroacetic acid, resorcinol, papain, podophyllotoxin, podophyllum resin, 5-fluorouracil, cantharone, a tar product, anthralin, benzoyl peroxide, squaric acid, imiquimod, and a combination of any two or more thereof.

19. The method of claim 1, wherein the subject is a child or adolescent.

20. The method of claim 1, wherein the subject is an adult.

21. The method of claim 1 wherein the heating device further comprises a layer of a non-therapeutic adhesive disposed on at least the peripheral zone of the heating device.

22. The method of claim 21 wherein the non-therapeutic adhesive is disposed on the peripheral zone and the central zone of the heating device.
